(19) United States
(12) Patent Application Publication
(10) Pub. No.: US 2008/0294210 A1
(43) Pub. Date: Nov. 27, 2008

(54) Leadless Implantable Cardioverter Defibrillator

(76) Inventor: Spencer Rosero, Pittsford, NY (US)

Correspondence Address: BLANK ROME LLP, 600 NEW HAMPSHIRE AVENUE, N.W., WASHINGTON, DC, 20037, US

(21) Appl. No.: 11/579,530

(22) PCT Filed: May 4, 2005
(86) PCT No.: PCT/US05/15379

§ 371 Date: Jun. 5, 2008

Related U.S. Application Data
(60) Provisional application No. 60/567,447, filed on May 4, 2004.
(60) Provisional application No. 60/567,449, filed on May 4, 2004.
(60) Provisional application No. 60/567,448, filed on May 4, 2004.

(51) Int. Cl. A61N 1/39

(52) U.S. Cl. 607/5

(57) ABSTRACT

A leadless implantable cardioverter defibrillator (5) for treatment of sudden cardiac death includes a controller and at least one remote module. The defibrillator does not require transvenous/vascular access for intracardiac lead placement. The controller is leadless and uses subcutaneous tissue in proximity of the chest and abdomen for both sensing and defibrillation. The controller and one or more remote sensors sense a need for defibrillation and wireless communicate with the controller. The controller and one of the sensors discharge a synchronized defibrillation pulse to the surrounding subcutaneous tissue in proximity to the heart.

### RELATED APPLICATIONS

The present application claims priority to provisional application Ser. Nos. 60/567,447, 60/567,448 and 60/567,449, each of which were filed on May 4, 2004.

### TECHNICAL FIELD

The present invention is generally related to cardiac defibrillators and, more particularly, is related to a method and an apparatus for providing a leadless implantable cardioverter defibrillator for the treatment of sudden cardiac death.

### BACKGROUND OF THE INVENTION

Defibrillation/cardioversion is a technique employed to counter arrhythmic heart conditions including some tachycardias in the atria and/or ventricles. Fibrillation is a condition where the heart has very rapid shallow contractions and, in the case of ventricular fibrillation, may not pump a sufficient amount of blood to sustain life. A defibrillator often is implanted in the chest cavity of a person who is susceptible to reoccurring episodes of ventricular fibrillation. Typically, electrodes are employed to stimulate the heart with electrical impulses or shocks, of a magnitude substantially greater than pulses used in cardiac pacing. The implanted defibrillator senses the rapid heart rate during fibrillation and applies a relatively high energy electrical pulse through wires connected to electrodes attached to the exterior wall of the heart.

Examples of pacemakers are shown, for instance, in U.S. Pat. Nos. 6,412,490 and 5,987,352. However, these technologies are hampered by the use of a transvenous lead for electrophysiologic stimulation. In those technologies, a transvenous/vascular access is required for the intracardiac lead placement. Those technologies are susceptible to an acute risk of cardiac tamponade, perforation of the heart or vasculature and long term risk of endocarditis or a need for intracardiac extraction of the lead due to failure. Also, current technologies present a problem for intracardiac defibrillation implantation in younger patients or in patients who are not candidates for the implantation because of anatomical abnormalities. Complex steps and risks are involved in obtaining venous vascular access and placement of the transvenous lead in the patient population requiring the defibrillation.

### SUMMARY OF THE INVENTION

Embodiments of the present invention provide an apparatus and method for a leadless implantable defibrillator for the treatment of sudden cardiac death. The defibrillator does not require transvenous/vascular access for intracardiac lead placement, but rather uses the subcutaneous tissue in the proximity of the chest and abdomen for both sensing and defibrillation.

In one approach, an implantable cardioverter defibrillator (ICD), configured to follow the abdominal contour, is located in the abdominal cavity. Two remote sensors, strategically placed in the upper torso area around the thorax, communicate with the ICD via radio frequency (RF) and analog tissue communication using subcutaneous tissue as a conducting medium. A conventional sensing algorithm utilized in the defibrillator includes capabilities to defibrillate as well as anti-tachycardia pacing. Anti-tachycardia therapy is possible for the detection of tachycardia rates that may be programmed into the ICD and vary between 100 bpm to 250 bpm. The defibrillator may also perform a pacemaker function and deliver cardiac pacing. However, all of the parameters for sensing and the type of desired stimulation (defibrillation, anti-tachycardia pacing, cardiac pacing) are programmable. A backside of the ICD includes a conductive surface for pacing and defibrillation via arrhythmia sensors/transducers.

In another approach, one of the remote sensors described above is replaced with a micro-thin patch with a lead connection to the ICD for a +/−polarity reversal implant. In yet another approach, ultrasonic signals are used to stimulate the heart as a back-up or as an adjunct to the electrical pacing that is provided. The ultrasonic signals could be used as an emergency pacing back-up. Antennae/transducers are located on the patient side of the device and include adjustable projection angles to provide the best acoustic angle.

Other systems, methods, features, and advantages of the present invention will be or become apparent to one with skill in the art upon examination of the following drawings and detailed description. It is intended that all such additional systems, methods, features, and advantages be included within this description, be within the scope of the present invention, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a perspective drawing of a preferred embodiment of the invention;

FIG. 2 is a rear view of the embodiment depicted in FIG. 1;

FIG. 3 is a perspective drawing of an embodiment of the invention using a microthin patch as a lead;

FIG. 4 is a diagram showing the energy from the defibrillation electrodes of the first remote module and the controller; and,

FIG. 5 is a circuit block diagram of the controller.

### DETAILED DESCRIPTION OF THE PREFERRED EMBODIMENT

In describing a preferred embodiment of the invention illustrated in the drawings, certain specific terminology will be used for the sake of clarity. However, the invention is not intended to be limited to that specific terminology, and it is to be understood that the terminology includes all technical equivalents that operate in a similar manner to accomplish the same or similar result.

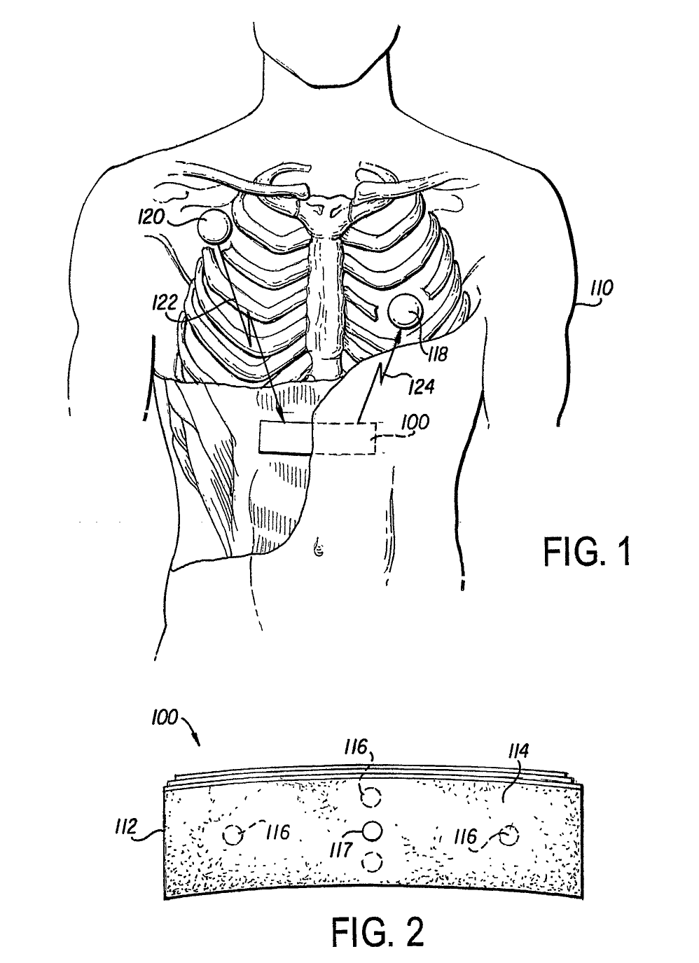

FIG. 1 shows a preferred embodiment of the implantable cardioverter defibrillator (ICD) 5. The defibrillator 5 includes a controller 100 and one or more satellite sensors 118, 120. The controller 100 is surgically implanted in the subcutaneous tissue in proximity to the chest and abdomen of a medical patient 110. The purpose of the ICD 5 is to produce an electrical stimulus or shock that either paces the heart or defibrillates the heart and returns the heart to a normal rhythm. The device 5 needs to be in close proximity to the target organ (here, the heart) in order to provide the highest amount of energy to be transmitted through the target organ. This reduces the amount of energy needed to be produced by the defibrillator 5 and minimizes the amount of energy expended to the surrounding tissue.

The controller 100 controls operation of the ICD 5, including operation of the satellite modules 118, 120. The back side 112 of the controller 100 includes a conductive surface 114 that operates as a defibrillation electrode by conveying an electrical signal or output (pulse) to the subcutaneous tissue that is used for defibrillation and pacing. The ICD produces energy outputs for cardioversion, whereby cardioversion shocks are synchronized to an underlying arrhythmia and range from 2-200 Joules and 24-2500V in biphasic waveform with and without adjustable waveform parameters. The energy delivered for defibrillation has a duration of about 4-40 ms. The total energy delivered per pulse is programmable so as to deliver a proportion of the total during the energy pulse. For biphasic and monophasic energy delivery, more than 50% of the energy is delivered during the first half of the total time during. The specific energy delivered is determined by the ability to defibrillate and return normal sinus rhythm. If sequential rapid shocks are used, then the energy per shock or pulse is expected to be in the range from about 24-400V.

Vector oriented electrodes/sensors 116 are dispersed throughout the back side 112 of the controller 100, to both sense a bioelectric signal which may indicate a need for a defibrillation, and to transmit a pacing voltage across the backside 112 and into the surrounding subcutaneous tissue. The electrodes 116 can either be dedicated to detection (sensing) a biologic signal and/or be used to transmit a pacing stimulus to the target organ. The electrodes 116 can also switch from sensing mode to a high voltage circuit that provides pacing and defibrillation.

A reference electrode 117 is provided on the front side of the controller 100 facing away from the heart (i.e., toward the skin), so that it is at a point farthest away from the defibrillation electrodes 114. The reference electrode 117 has a high impedance and a polarity that is opposite that of the conductive surface 114, so that the reference electrode 117 operates as a ground. Accordingly, the conductive surface 114 forms a circuit with the heart and the reference electrode 117. The conductive surface 114 generates a defibrillation pulse that is transmitted to the heart and is grounded by the reference electrode 117.

A first satellite sensor and/or stimulation module 118 is implanted in the subcutaneous tissue in positions around the thorax such as the left and posterior area of the chest. The first module 118 is configured in the same manner as the controller 100, with a conductive surface facing the heart which is used for imparting a defibrillation pulse and a reference electrode facing away from the heart that forms a ground for the module's conductive surface. The controller 100 and module 118 are positioned so that the heart is located between them, with the controller 100 on the front of the patient, and the first module 118 on the posterior of the patient.

Turning to FIG. 4, the energy fields 200, 210 for the defibrillation electrode 114 of the controller 100 and the defibrillation electrode of the first module 118 are shown, respectively. As shown, the defibrillation electrode 114 of the controller 100 and the defibrillation electrode of the first module 118 are positioned so that their respective energy fields 200, 210 envelope the heart. This imparts a stimulation to the heart to obtain the best heart rhythm signal with the least amount of electrical noise, maximize the signal-to-noise ratio, and provide an energy field that maximizes the amount of defibrillation energy passing through the heart.

This first module 118 has two important functions, namely to record and transmit biologic information such as the rhythm that the heart is in, and to provide an electrode (cathode and/or anode) pole needed to provide defibrillation of the heart. The polarity of the controller 100 and the module 118 is switched by the primary controller 100. The loop for the electrical defibrillation shock is completed by using the subcutaneous tissue as the conductor to electrically connect both the controller 100 and the module 118. The defibrillation shock energy is simultaneously charged to capacitors which are located in each of the controller 100 and the first module 118 with a fixed or variable capacitance of 25-350 μF. However, a capacitor need not be provided where the waveforms can be generated using a battery. The shock energy is synchronized via wireless communications between the controller 100 and the first module 118. The ICD 5 is capable of imparting a shock pulse to the patient of 2-300 Joules biphasic or up to 100 J for rapid pacing.

The defibrillation electrodes of the controller 100 and the first module 118 (which is located on the posterior of the patient) have different +/−polarities (as best shown in FIG. 4), which are assigned by the controller 100. The energy released by the electrodes of the first module 118 can be controlled to deliver a different energy (in terms of total energy, waveform (polarity, voltage amplitude, single/multiple pulses and time dependency) than the high impedance external surface electrodes 116 of the controller 100. The impedance is about 20-90 ohms for the controller 100 and the first module 118. The controller electrodes 116 provide for the shunting or transfer of energy through body tissue in order to allow a closed circuit between the controller 100 and the first module 118.

The controller electrodes 116 are about 1-10 cm away from the electrodes of the first module 118. The positioning of the controller 100 and first module 118 maximizes the amount of energy going through the heart and minimizes the energy lost through the tissue. The energy loss can be further improved by adding a connecting cable between the defibrillation electrodes of the controller 100 and the first module 118 to ground the device and complete the circuit between those components. Reference electrodes 117 is located on the controller 100 at the farthest point away from the defibrillation electrode 114 and will have a significantly less surface area and greater impedance compared to the defibrillation electrode.

The surface electrodes 116 optionally deliver energy slowly through the body tissue after they had been rapidly dumped into a separate capacitor thus allowing the movement of current to complete the circuit. The rapid dump provides for the majority of the energy to go through the heart with the circuit being completed using the subcutaneous tissue. Whatever energy does not go to the heart is absorbed by a capacitor and not the tissue. The capacitor can then slowly release the energy into the tissue in a harmless manner. The primary controller 100 and first satellite module 118 location are determined by the targeted physiologic signal/stimulus and the defibrillation efficacy at that site (i.e., the site that requires the least amount of energy to defibrillate the heart).

The first satellite module 118 senses the biological logistics of the surrounding subcutaneous tissue as well as the target organ (e.g., the heart), converts those biological logistics to an analog signal and transmits the signal to the controller 100 using either radio frequency and/or direct electrical signal that is transmitted using the body's native subcutaneous tissue as the conductor. The signaling methods may be integrated to provide redundancy and increase signal quality. The sensed signals will include sensing heart rhythms (electrocardiographic) signals to sense the biologic activity of interest. The communication protocol between devices will use either radio frequency and/or subcutaneous analog methods. There is also the option of using a hardwired approach between predetermined sensors to other devices within the whole implanted system. This may be via fiber optic transmission or standard metallic conductors wiring.

A second satellite sensor module 120 is preferably provided only to enhance sensing of the patient conditions, and is not used for stimulation. The module 120 is implanted in the subcutaneous tissue in positions around the thorax such as the right upper quadrant area of the chest. The site is determined by the signal to noise ratio and is usually a distance from the heart that is determined by the individual patient anatomy. This can be mapped during the implantation itself and/or using external sensing patches as determined, for instance by the use of temporary self adhesive electrodes positioned around the torso before the implantation procedure during which the heart's electrical signal is measured (ECG, the QRS part of the electrocardiogram which represents depolarization of the heart).

The position of the temporary mapping electrode that provides the greatest amplitude of the signal is chosen as allowing for optimal energy delivery for the first module 118 and controller 100. The second satellite module 120 is placed remote from this the controller 100 and the first module 118 at a site that is determined by the clearest ECG signal obtained after mapping the surrounding tissue. The sensor 120 converts those biological logistics (such as the electrical heart rhythm and other biologic signals such as minute ventilation, oxygen saturation, pH) to a signal and transmits the signal to the controller 100 using either wireless radio frequency or ultrasonic methods, or a hardwired fiber optic or metallic conductor.

After mapping, the anterior controller 100 is placed at the front thorax. An anterior position is chosen that will place the heart ventricle between the controller 100 and the first module 118 that provides maximum exposure to the energy delivered by the electrodes for those devices. The incision can be made to the subcutaneous tissue and dissection made within the surgical pane over the intercostals/rib section that meets the minimum diameter of the device. The controller 100 may also be placed in the upper abdomen if that site provides a better signal and vector for defibrillation in an individual. The controller 100 is then molded (or it can have a fixed shape) and placed within the site with the defibrillation electrodes 114 and the reference electrode 117.

The first module 118 is then positioned. If the patient has a small thorax, the same incision can be used to position the first module 118. A tunneling device can be used with the module 118 affixed at its distal end. The device 118 is tunneled to the posterior or posterolateral region which was marked during mapping. After the cardiac signal is confirmed as adequate and wireless communication established with the controller 100, the module 118 is released. IF the same incision cannot be used, a second incision can be made closer to the final site. Finally, the second module 120 is inserted to a subcutaneous position through an incision in the right anterior chest. However, the module 120 can be implanted at any other location that provides a good cardiac signal and where wireless communication can be established with the controller 100.

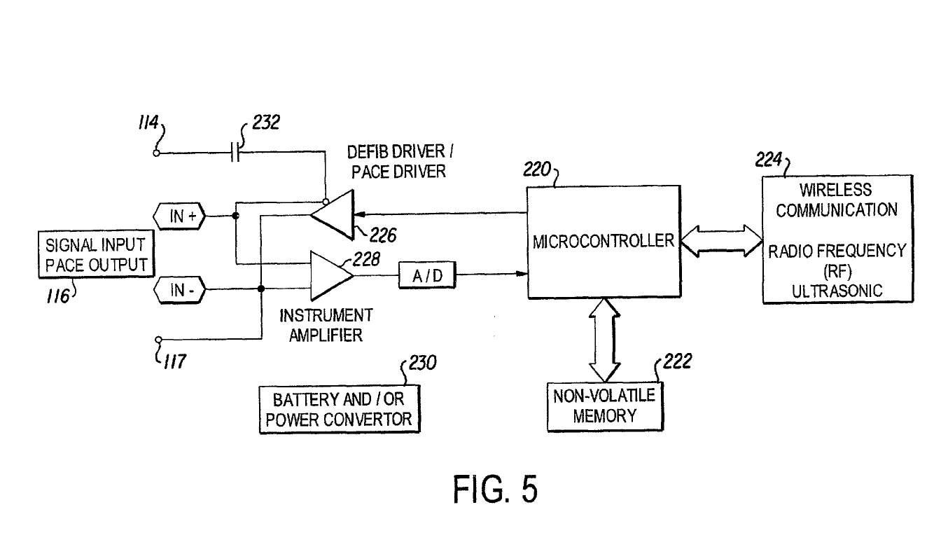

Turning to FIG. 5, a circuit diagram for the controller 100 is shown. The controller 100 generally includes a processor or microcontroller 220, memory 222, wireless communication device 224, defibrillation/pace driver 226, amplifier 228 and power supply 230. The processor 220 also receives signals from the remote modules 118, 120 and the electrodes/sensors 116 to sense various patient conditions. Based on those signals, the processor 220 then determines whether or not a defibrillation or other action needs to be taken. The processor 220 then outputs a control signal to the defibrillation electrode 114 of the controller 100 and to the remote modules 118, 120, via communication device 224 that synchronizes the application of a defibrillation pulse. The processor 220 can also output a control signal to the electrodes 116 to generate a pacing pulse.

The processor 220 also controls the type of sensing performed by the electrodes/sensors 116. The wireless communication device 224 can be, for instance, a radio frequency or ultrasonic transceiver, but can also be hardwired if necessary. The power supply 230 can either be a battery and/or a power converter, or a inductive power coil that receives power from a remote device that transmits RF energy. The amplifier 228 reduces electrical signal artifact during sensing of physiologic signals and amplifies the signal prior to digitization by an A/D converter. The capacitor 232 stores power after step up of the voltage in order to provide a single high voltage defibrillation pulse on command. The pace driver 226 sets the timing, amplitude and duration of the pacing pulse, which is a low voltage pulse sent to the heart module 118 to generate a pacing pulse.

The microprocessor 220 can also record the electrical signals corresponding with the heart rhythm in memory 222. Preferably, the sensed signals are analyzed at the controller 100. Those signals instead, or also, can be analyzed by a processor provided at the satellite modules 118, 120. The first and second modules 118, 120 have similar circuits to that shown for the controller 100. However, the first and second modules 118, 120 need not have a microcontroller 220 or memory 222, unless it is used to perform an analysis on the conditions sensed by its sensors.

The more sensor information available from the different sites, the higher the specificity and sensitivity of detecting the true heat rhythm signal. The analog signal conveying the biological logistics of the heart condition such as QRS, atrial P waves, QRS frequency, QT interval, R-R intervals, R-R variability, etc. is communicated to the controller 100 via a wireless signal 122, preferably as a radio frequency signal. The first and second modules 118, 120 can be programmed to record and transmit signals to the controller 100 continuously or in an intermittent fashion.

The communication device 224 includes an antenna is located in the controller 100 and each of the first and second satellite sensors 118, 120 to promote the radio frequency communication therebetween. The antenna transmits and receives RF or ultrasonic signals. The antenna can also be placed in contact with the subcutaneous tissue to transmit frequency modulation signals to/from the sensors 118, 120 using the subcutaneous tissue as a medium. The communication device 224 of the controller 100 has a transmitter that transmits a radio frequency signal 124 to the first satellite sensor 118 in order to communicate with that sensor, in response to detecting the abnormal heart rhythm signal when defibrillation of the heart is required. The sensors 118, 120 transmits patient condition information to the controller 100, which determines whether there is an abnormality. The controller 100 transmits control signals to coordinate the delivery of energy and stimulus imparted by the controller 100 and the first module 118. A central processing unit (CPU) 220 in the controller 100 coordinates the receipt of the need for defibrillation, and the transmission of the defibrillation pulse. If a defibrillation pulse is determined necessary, the defibrillation or pacing pulse includes a range of 0.25-100 msec with variable or programmable portions of the delivered energy being delivered within the biphasic waveform per unit time.

The transmitted radio frequency signal 124 from the controller 100 is received by an electronic circuit via a radio frequency detector 224 in the first satellite sensor 118. The electronic circuit includes a capacitor (not shown), or similar element which is charged using energy from the radio frequency signal 124. A discharging circuit discharges the capacitor to apply a voltage across the surrounding subcutaneous tissue, thus initiating a defibrillation pulse. The conductive surface 114 in the back side 112 of the controller 100 is vector oriented so that the energy imparted is directed to the heart. The conductive surface 114 simultaneously conveys the defibrillation pulse with the conductive surface in the first module 118 to the heart. The conductive surface 114 of the back side 112 as a broadening medium to disperse the defibrillation pulse. The surface area is increased near the target organ so that the electrical field is greatest around the target organ. The ICD also includes circuitry for sensing bradycardic rhythm.

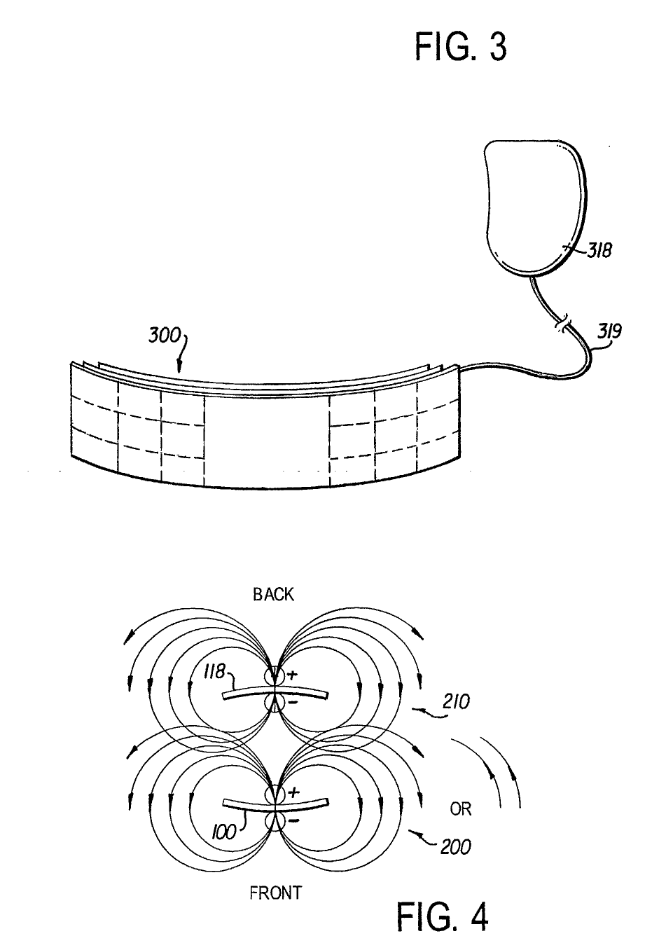

In FIG. 3, an optional microthin patch 318 is provided when the energy fields created by the controller 100 and the first module 118 are insufficient, such as when the reference electrode 117 is unable to close the circuit to provide energy flow through the heart. The patch 318 is placed under the skin at the lateral aspect of the chest at the level of the heart. The patch 318 extends the energy field of the conductive surface 114 of the controller 100 and the first module 118. The microthin patch 318 is electrically connected to the controller 300 via a wire lead 319. The lead wire 319 operates to complete the circuit between the conductive surfaces 114 of the controller 100 and module 118. The advantage is that parts of the system are wireless. However, where there are increased defibrillation thresholds (amount and waveform characteristics of energy required to defibrillate), the energy required and/or waveform of the shock needs to be changed, there is an option to connect a wire for grounding purposes from the controller 100 to the first module 118. In addition, all communication and control is wireless. The embodiment of FIG. 3 may be used, for instance, where the defibrillation threshold is high and the subcutaneous transmission is inadequate to generate the energy required for a defibrillation pulse.

The primary purpose of the controller 100 is to communicate with the sensor modules 118, 120. Since the modules 118, 120 must be much smaller in size in order to be positioned about the target organ, they have limited microprocessor capabilities. The second module 120 also does not have to be within the energy delivery field that encompasses the heart for defibrillation. Accordingly, the second module 120 may be placed outside the shock energy field if they have other functions, such as monitoring other physiologic signals and verifying what the controller 100 is seeing.

The sensor modules 118, 120 can also communicate with one another to verify the signals being recorded from different angles or electrocardiographic vectors. The sensor modules 118, 120 placed at various location provide different views of the same signal and thus different information. There are at least two sensors (controller 100 and module 118) to perform sensing of the patient conditions, and preferably the third sensor (module 120) is used to provide enhanced sensing. However, any number of sensors can be provided.

In yet another embodiment, a transducer can be provided in the controller 100 and the first modules 118 to generate ultrasonic signals used to stimulate the heart as a back-up or as an adjunct to the electrical pacing that is provided. The ultrasonic signals are used as an emergency pacing back-up. Antennae/transducers are located on the patient side of the controller 100 and include adjustable projection angles from 30-120 degrees to provide the best acoustic angle that is able to trigger a heartbeat or stimulate the heart. The transducers can be used instead of, or in addition to, the electrodes 116.

In addition, the transducer can be utilized to generate an acoustic/ultrasound signal for communication between the controller 100 and the modules 118, 120. The transducers in the controller 100 and module 118 also operates as a sensor to detect cardiac dynamics. The acoustic/ultrasound signaling system detects cardiac motion and correlates the active beating of the heart and/or blood flow using Doppler signals with electrophysiologic body signals. This enables the defibrillator 5 to electrically and mechanically confirm that the heart is functioning.

The controller 100 and/or satellite modules 118, 120 can be constructed as described in co-pending application number PCT/______, entitled “Implantable Bio-ElectroPhysiologic Interface Matrix,” filed herewith claiming priority to Ser. No. 60/567,448, filed May 4, 2004, and/or co-pending application number PCT/______, entitled “Leadless Implantable Intravascular Electrophysiologic Device for Neurologic and Cardiovascular Sensing and Stimulation,” filed herewith claiming priority to Ser. No. 60/567,447, filed May 4, 2004. The contents of each of these applications is incorporated herein by reference.

It should be emphasized that the above-described embodiments of the present invention, and particularly, any preferred embodiments, are merely possible examples of implementations, merely set forth for a clear understanding of the principles of the invention. Many variations and modifications may be made to the above-described embodiments of the invention, without departing substantially from the spirit and principles of the invention. All such modifications and variations are intended to be included herein within the scope of this disclosure and the present invention and protected by the following claims.

1. A leadless implantable defibrillator comprising a controller having a controller sensor for sensing patient conditions, a controller electrode for imparting a stimulation to the patient, and a controller wireless communicator, and further comprising at least one remote module having a remote sensor, a remote electrode, and a remote wireless communicator for wirelessly communicating with the controller wireless communicator.

2. The defibrillator of claim 1, wherein said remote wireless communicator and said controller wireless communicator communicate with one another using subcutaneous tissue as a communication medium.

3. The defibrillator of claim 1, wherein the controller wireless communicator comprises a wireless transmitter and the remote wireless communicator comprises a wireless receiver, wherein said wireless transmitter wirelessly transmits a signal to said wireless receiver.

4. The defibrillator of claim 1, wherein said controller is located in subcutaneous tissue in proximity to the chest and abdomen and said at least one remote module is located in the subcutaneous tissue.

5. The defibrillator according claim 1, wherein at least two remote modules are positioned, subcutaneously, around the thorax of the subject and communicate via radio frequency signals with the defibrillator.

6. The defibrillator according to claim 1, wherein the controller sensor and the remote sensor communicate sensed information to the controller and the controller determines whether there is a need for defibrillation.

7. The defibrillator according to claim 1, wherein the controller includes a first antennae.

8. The defibrillator according to claim 7, wherein the at least one remote module includes a second antennae.

9. A cardiac defibrillator comprising: a controller implanted in the subcutaneous tissue of a patient in proximity to the subject's chest and abdomen, a remote module implanted in the subcutaneous tissue of the patient, said controller having a wireless transmitter for wirelessly transmitting a signal from said controller to said remote module.

10. The defibrillator according to claim 9, wherein said defibrillator is leadless.

11. The defibrillator according to claim 9, wherein the signal is transmitted using the subcutaneous tissue as a communication medium.

12. The defibrillator according to claim 9, wherein the signal is transmitted via radio frequency.

13. The defibrillator according to claim 9, wherein said remote module includes a sensor for sensing a patient condition.

14. The defibrillator according to claim 9, wherein said remote module includes an electrode for imparting a defibrillation pulse to the patient.

15. A method for defibrillating the heart of a patient, the method comprising:
implanting a controller in the subcutaneous tissue in proximity to the chest and abdomen;
implanting a remote module subcutaneously in a posteriolateral location in the left chest area of the patient;
sensing a patient condition at the remote module and at the controller;
wirelessly transmitting the patient condition from the remote module to the controller;
determining at the controller when defibrillation of the heart is required based on the sensed patient conditions;
wirelessly transmitting a defibrillation signal from the controller to the remote module in response to determining when defibrillation of the heart is required; and
applying a defibrillation pulse by the controller and the remote module in response to receiving the defibrillation signal.

16. A leadless implantable apparatus for the treatment of sudden cardiac death of a subject wherein the subcutaneous tissue in proximity to the chest and abdomen is used for both sensing and defibrillation, comprising:
a controller located in the subcutaneous tissue;
a remote module located subcutaneously in the upper right quadrant of the subject's chest and in radio frequency communication with the controller; and
a microthin patch located in a posteriolateral position in the upper left quadrant of the chest with an electrical wire connected to the controller.

17. The apparatus according to claim 16, wherein the microthin patch includes an electronic circuit for applying a voltage across the subcutaneous tissue in response to an electrical signal from the defibrillator.
